(19) United States
(12) Patent Application Publication
(10) Pub. No.: US 2017/0290553 A1
(43) Pub. Date: Oct. 12, 2017

(54) SENSOR SYSTEM

(71) Applicant: Renesas Electronics Corporation, Tokyo (JP)

(72) Inventors: Masaharu Matsudaira, Tokyo (JP); Takashi Hase, Tokyo (JP); Akira Tanabe, Tokyo (JP); Kazuya Uejima, Tokyo (JP)

(21) Appl. No.: 15/468,063

(22) Filed: Mar. 23, 2017

(30) Foreign Application Priority Data
Apr. 8, 2016 (JP) 2016-078202

(51) Int. Cl. A61B 5/00; A61B 5/021; A61B 5/11; A61B 5/01

(52) U.S. Cl. CPC A61B 5/7285; A61B 5/01; A61B 5/021; A61B 5/0002; A61B 5/1118; A61B 5/742; A61B 2560/0252; A61B 2562/029; A61B 2560/0475; A61B 2560/0209; A61B 2560/0431

(57) ABSTRACT

In order to appropriately set a condition for measurement of a sensor for measuring an object to be measured in accordance with a change of an external index that can affect the object to be measured, a sensor system includes first and second sensors, a determination unit for outputting a detection signal when a measurement result of the first sensor satisfies a predetermined condition, a measurement condition storage unit for storing a condition for measurement of the second sensor, and a control unit for performing measurement by the second sensor separately from measurement in accordance with the condition for measurement, when having received the detection signal, and for updating the condition for measurement of the second sensor stored in the measurement condition storage unit based on a result of the performed measurement.

### CROSS-REFERENCE TO RELATED APPLICATIONS

The disclosure of Japanese Patent Application No. 2016-078202 filed on Apr. 8, 2016 including the specification, drawings and abstract is incorporated herein by reference in its entirety.

### BACKGROUND

This disclosure relates to a sensor system, and more particularly relates to a sensor system including a plurality of sensors.

In development of IoT (Internet of Things), it is an important issue how to collect data from sensor terminals present in everywhere in society. In particular, in a case where the sensor terminal has a limitation on a supplied power, for example, is driven by a harvesting power source or a battery, a time and/or a frequency of measurement by the sensor is limited, and therefore data acquisition has to be performed efficiently.

As for a technique of efficiently acquiring data by the sensor, Japanese Unexamined Patent Application Publication No. 2010-057552 discloses a configuration that accumulates measurement values with regard to a plurality of items of biological indices, obtains a change pattern of each biological index or a relation between the biological indices, determines, when a value out of the change pattern is measured for a certain biological index, a type of a biological index required for evaluation of a health condition, and a measurement timing, and a number of measurements of that biological index, for example, based on a relation with another biological index, and presents those to a user.

### SUMMARY

However, the technique disclosed in Japanese Unexamined Patent Application Publication No. 2010-057552 merely presents the determined measurement timing to the user, but an actual measurement timing depends on the user. Further, that technique determines the timing of the measurement by the sensor based on a change of the user's state, that is, a change of an object to be measured, but is independent of the object to be measured and does not consider a change of an external index that can affect the object to be measured.

The present disclosure has been made for solving the above problems. It is an object in an aspect to provide a sensor system that can appropriately set a condition of measurement for an object to be measured in accordance with a change of an external index that can affect the object to be measured.

Other problems and novel features will be apparent from the description of the present specification and the accompanying drawings.

According to an embodiment, a sensor system includes first and second sensors, a determination unit for outputting a detection signal when a measurement result of the first sensor satisfies a predetermined condition, a measurement condition storage unit for storing a condition for measurement of the second sensor, and a control unit for performing a measurement by the second sensor separately from measurement in accordance with the condition for measurement when having received the detection signal, and for updating the condition for measurement of the second sensor stored in the measurement condition storage unit based on measured data.

A sensor system according to an embodiment can appropriately set a condition for measurement for an object to be measured in accordance with a change of an external index that can affect the object to be measured.

### BRIEF DESCRIPTION OF THE DRAWINGS

FIG. 1 illustrates an outline of a sensor system according to embodiments.

FIG. 2 illustrates an outline of a system configuration of a sensor system according to a first embodiment.

FIG. 3 illustrates a configuration example of the sensor system according the first embodiment.

FIG. 4 is a flowchart illustrating control of an environment monitoring system according to the first embodiment.

FIG. 5 is a flowchart illustrating control of a health care system according to the first embodiment.

FIG. 6 is a flowchart illustrating control that updates a condition of measurement by a biosensor according to the first embodiment.

FIG. 7 illustrates causes of heat stroke.

FIG. 8 illustrates another control example in Step S66 in FIG. 6.

FIG. 9 illustrates a configuration example of a sensor system according to a second embodiment.

FIG. 10 is a flowchart illustrating control of a health care system according to the second embodiment.

FIG. 11 illustrates control for updating a condition of measurement by an environment sensor according to the second embodiment.

FIG. 12 illustrates a configuration example of a sensor system according to a third embodiment.

FIG. 13 illustrate an example of use of the sensor system according to the third embodiment.

### DETAILED DESCRIPTION

Embodiments of the present invention are described in detail below, with reference to the drawings. The same or similar portions are labeled with the same reference signs, and the redundant description is omitted.

### A. Outline

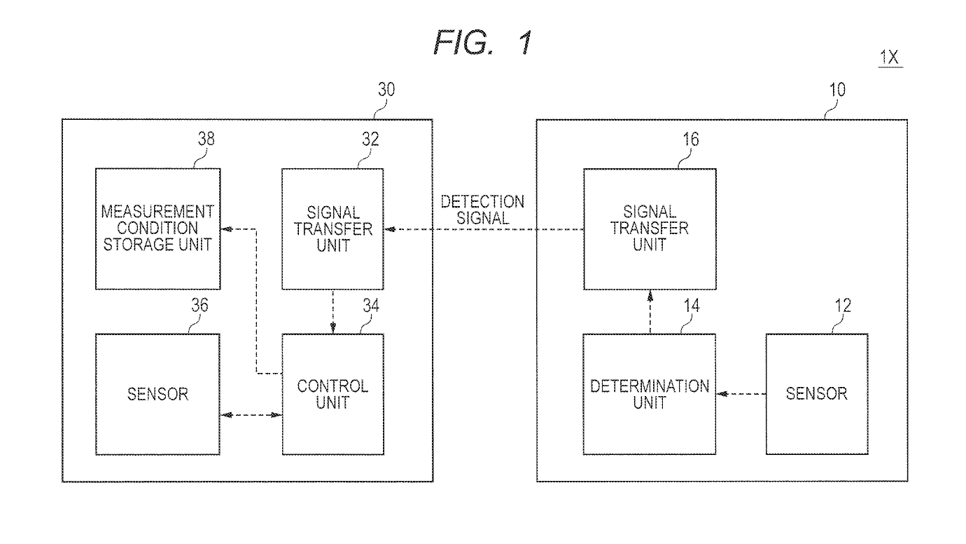

FIG. 1 illustrates an outline of a sensor system 1X according to embodiments. Referring to FIG. 1, the sensor system 1X includes a sensing system 10 and a sensing system 30. The sensing system 10 includes a sensor 12, a determination unit 14, and a signal transfer unit 16. The sensing system 30 includes a signal transfer unit 32, a control unit 34, a sensor 36, and a measurement condition storage unit 38. The sensing systems 10 and 30 is configured to perform operations, such as measurement, independently of each other during a normal operation, but operate together when a predetermined condition described later is satisfied.

The sensor 12 measures an external index (e.g., a temperature) and outputs the measurement result to the determination unit 14. The determination unit 14 determines whether the measurement result of the sensor 12 satisfies a predetermined condition, and transmits a detection signal to the sensing system 30 via the signal transfer unit 16 when that condition is satisfied.

The control unit 34 performs measurement for an object to be measured with the sensor 36 in accordance with a condition for measurement (for example, measurement is performed every three minutes) stored in the measurement condition storage unit 38. When having received the detection signal from the sensing system 10 via the signal transfer unit 32, the control unit 34 performs a measurement with the sensor 36 separately from the condition for measurement stored in the measurement condition storage unit 38. Based on the result of that measurement, the control unit 34 updates the condition for measurement stored in the measurement condition storage unit 38. For example, the control unit 34 updates the condition for measurement stored in the measurement condition storage unit 38 to increase a measurement frequency, when the result of that measurement shows an abnormal value.

According to the above, the sensor system 1X according to the embodiments can measure a change of the external index that can affect the object to be measured, and can appropriately set the condition for measurement of the object to be measured based on a state of the object to be measured in accordance with this change of the external index. A configuration and control for achieving this sensing system are described below.

### B. First Embodiment

### b1. Overall Configuration of Sensor System 1

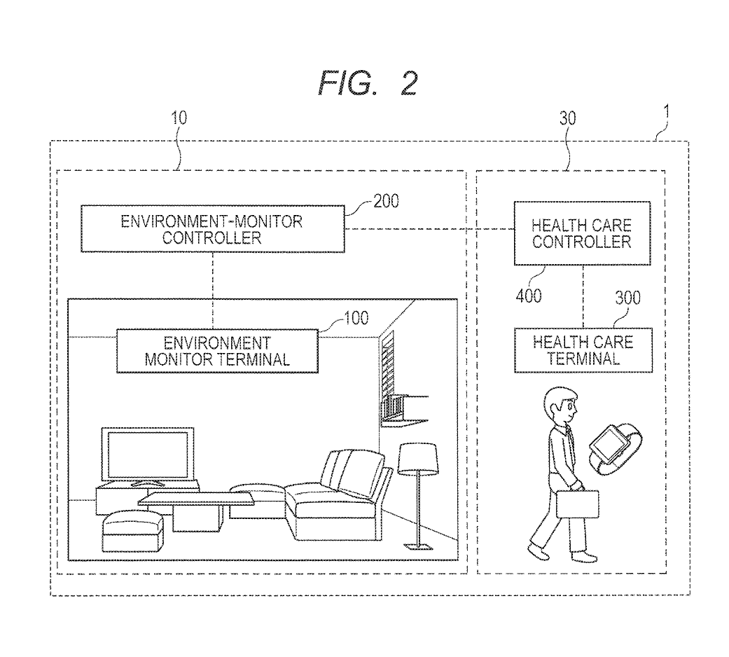

FIG. 2 illustrates an outline of a system configuration of a sensor system 1 according to a first embodiment. Referring to FIG. 2, the sensor system 1 includes an environment monitoring system 10 for measuring an external index and a health care system 30 for measuring an object to be measured. In the first embodiment, the external index is environment information, such as a temperature or a humidity, for example. Further, in the first embodiment, the object to be measured is biological information, such as body temperature or blood pressure. Because a change of an external environment affects a living body, the environment information and the biological information are relevant to each other for a certain time period.

The environment monitoring system 10 includes an environment monitor terminal 100 for acquiring environment information and an environment-monitor controller 200 for controlling the environment monitor terminal 100. The health care system 30 includes a health care terminal 300 for acquiring biological information and a health care controller 400 for controlling the health care terminal 300.

### b2. Configuration of Sensor System 1

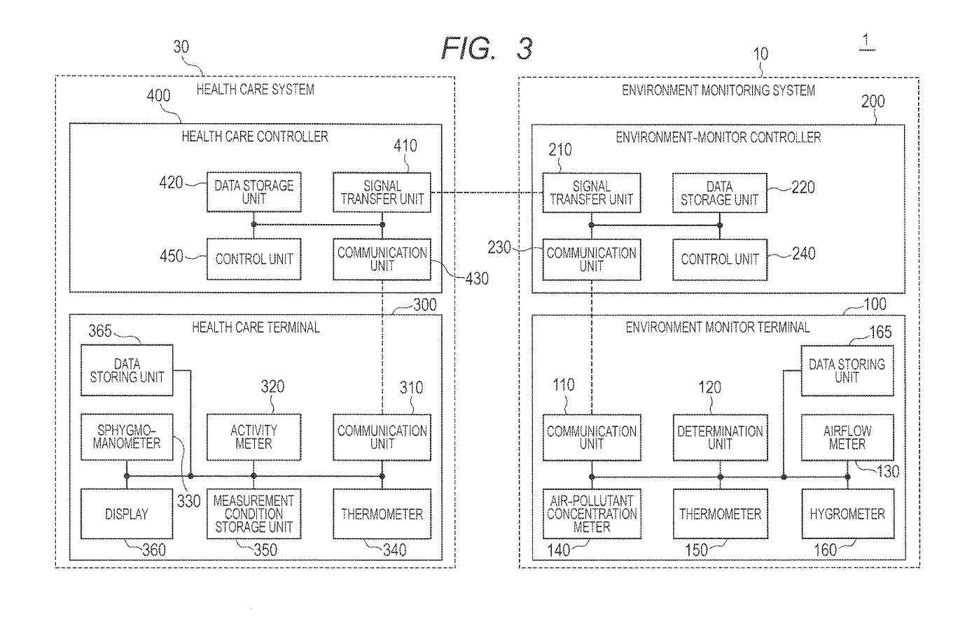

FIG. 3 illustrates a configuration example of the sensor system 1 according to the first embodiment. Referring to FIG. 3, the environment monitor terminal 100 includes a communication unit 110, a determination unit 120, an airflow meter 130, an air-pollutant concentration meter 140, a thermometer 150, a hygrometer 160, and a data storing unit 165. The communication unit 110 is configured to be communicable with the environment-monitor controller 200. In the first embodiment, the environment monitor terminal 100 acquires environment information including an airflow, a concentration of air pollutants, a temperature, and a humidity, as an example.

The airflow meter 130, the air-pollutant concentration meter 140, the thermometer 150, and hygrometer 160 for acquiring the environment information (hereinafter, also collectively referred to as an “environment sensor”) output measurement results to the determination unit 120 and the data storing unit 165. The determination unit 120 determines whether the environment information received from the environment sensor satisfies a predetermined condition, and outputs a detection signal to the environment-monitor controller 200 via the communication unit 110 when having determined that the received environment information satisfies that condition. The data storing unit 165 is a storing unit for temporarily storing the environment information. When data stored in the data storing unit 165 has reached a predetermined amount, the environment monitor terminal 100 outputs the data to the environment-monitor controller 200.

The environment monitor terminal 100 is preferably attached and fixed inside or around a building in which a user carries out daily activities.

The environment-monitor controller 200 includes a signal transfer unit 210, a data storage unit 220, a communication unit 230, and a control unit 204. The signal transfer unit 210 is configured to be communicable with the environment monitoring system 10. The data storage unit 220 stores environment information received from the environment sensor via the communication unit 230. The control unit 240 manages an overall operation of the environment-monitor controller 200 based on the environment information stored in the data storage unit 220, for example. The environment-monitor controller 200 outputs a control instruction for controlling the environment sensor to the environment monitor terminal 100 in accordance with a condition for measurement determined by the control unit 240. Processing of sensor data described later is performed by a data processing unit (not illustrated).

The health care terminal 300 includes a communication unit 310, an activity meter 320, a sphygmomanometer 330, a thermometer 340, a measurement condition storage unit 350, a display 360, and a data storing unit 365. A power is supplied to each device mounted in the health care terminal 300 from a battery (not illustrated). An example of the health care terminal 300 is a wristband type terminal and is configured to be wearable on a user's body. That is, the health care terminal 300 is configured to be movable together with user's movement. A form of the health care terminal 300 may be any form, such as an adhesive bandage type, as long as it is easily wearable. In a case where the health care terminal 300 is an adhesive bandage type terminal, the sphygmomanometer 330 measures blood pressure without being worn around an arm of the user (for example, optically).

The communication unit 310 is configured to be communicable with the health care controller 400. In the first embodiment, the health care terminal 300 acquires biological information including an amount of activity, blood pressure, and body temperature, as an example. The activity meter 320, the sphygmomanometer 330, and the thermometer 340 for acquiring the biological information (hereinafter, also collectively referred to as a “biosensor”) output measurement results to the display 360 and the data storing unit 365. The display 360 presents the biological information input from the biosensor to the user. The data storing unit 365 is a storing unit for temporarily storing the biological information. When a predetermined condition is satisfied, the health care terminal 300 outputs the biological information stored in the data storing unit 365 to the health care controller 400 via the communication unit 310. The measurement condition storage unit 350 stores various conditions for measurement by the biosensor. It is desirable that the measurement condition storage unit 350 and the data storing unit 365 are non-volatile memories that have a low power consumption and allow an random access thereto, such as a ReRAM (Resistive Random Access Memory), because there is a possibility that they frequently repeat an operation (read and write) and stop.

The health care controller 400 includes a signal transfer unit 410, a data storage unit 420, a communication unit 430, and a control unit 450. The signal transfer unit 410 and the communication unit 430 are configured to be respectively communicable with the signal transfer unit 210 and the communication unit 310. The data storage unit 420 stores biological information received from the biosensor via the communication unit 430. The control unit 450 manages an overall operation of the health care controller 400. The control unit 450 updates a condition for measurement stored in the measurement condition storage unit 350 (hereinafter, also referred to as a “condition for measurement of the biosensor”) when a predetermined condition is satisfied. Processing of sensor data described below is performed by a data processing unit (not illustrated).

Signal transmission and reception between the communication unit 110 and the communication unit 230, between the communication unit 310 and the communication unit 430, and between the signal transfer unit 210 and the signal transfer unit 410 is preferably performed wirelessly, but may be performed in a configuration in which they are coupled by wire. Next, an operation of the environment monitoring system 10 is described.

### b3. Operation of Environment Monitoring System 10

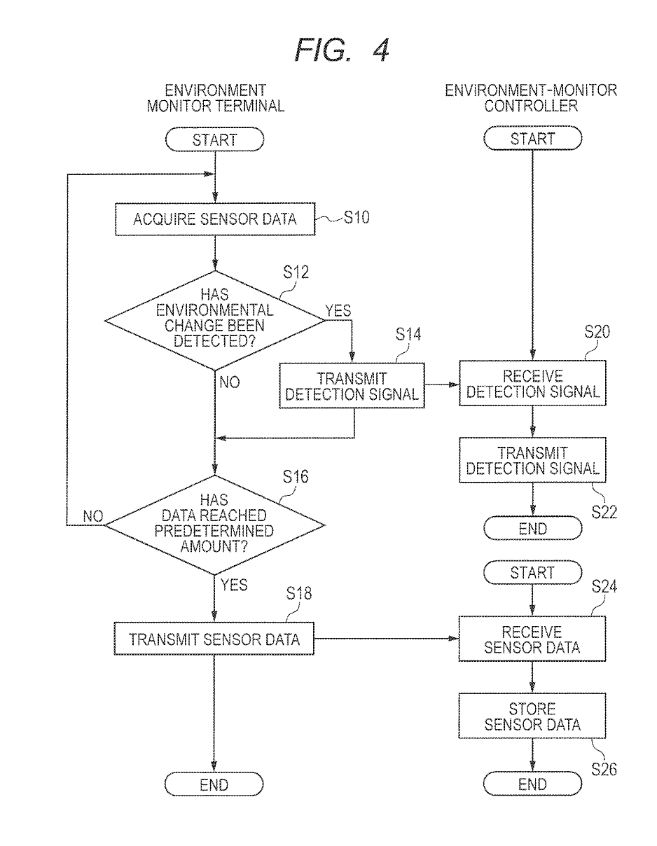

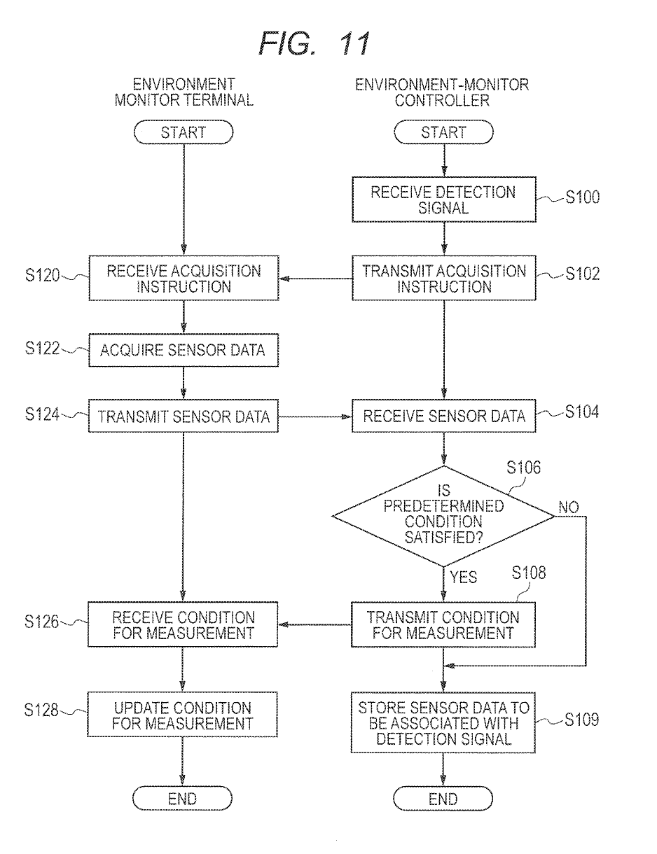

FIG. 4 is a flowchart of control of the environment monitoring system 10 according to the first embodiment. A process illustrated in FIG. 4 is achieved by execution of a control program by the environment monitor terminal 100 and the environment-monitor controller 200. In another aspect, the process may be performed partially or entirely by a circuit element or other hardware. This can be also applied to FIG. 11 described later.

Referring to FIG. 4, in Step S10, an environment sensor acquires environment information and outputs the environment information to the determination unit 120 and the data storing unit 165 in accordance with a control signal from the environment-monitor controller 200. In Step S12, based on the acquired environment information, the environment monitor terminal 100 determines whether a change of the environment information has been detected. More specifically, the determination unit 120 determines whether the acquired environment information satisfies a predetermined condition.

When having determined that the acquired environment information satisfies the predetermined condition (YES in Step S12), the environment monitor terminal 100 transmits a detection signal to the environment-monitor controller 200 via the communication unit 110. When having received the detection signal from the environment monitor terminal 100 in Step S20, the environment-monitor controller 200 transmits the received detection signal to the health care system 30 in Step S22. The processes in Steps S20 and S22 in the environment-monitor controller 200 are so-called interrupt processing, and are different from a normal process. The environment-monitor controller 200 transmits the detection signal in Step S22, and then ends a series of interrupt processing. Thereafter, the environment-monitor controller 200 returns to the normal process and waits for reception of the environment information from the environment monitor terminal 100.

Meanwhile, when having determined that the acquired environment information does not satisfy the predetermined condition (NO in Step S12), the environment monitor terminal 100 proceeds to Step S16. In Step S16, the environment monitor terminal 100 determines whether an amount of sensor data (environment information) stored in the data storing unit 165 has reached a predetermined amount. The predetermined amount is 256 kB, for example.

When having determined that the amount of the data stored in the data storing unit 165 has reached the predetermined amount (YES in Step S16), the environment monitor terminal 100 transmits the stored sensor data (environment information) to the environment-monitor controller 200 (Step S18). Due to this configuration, the environment monitor terminal 100 no longer has to communicate with the environment-monitor controller 200 every time it measures the environment information, so that a power consumption can be suppressed.

In Step S24, the environment-monitor controller 200 receives the environment information transmitted from the environment monitor terminal 100. In Step S26, the environment-monitor controller 200 stores the received environment information in the data storage unit 220. On the other hand, when having determined that the amount of the data stored in the data storing unit 165 has not reached the predetermined amount (NO in Step S16), the environment monitor terminal 100 returns the process to Step S10, and waits for input of the control signal from the environment-monitor controller 200 again. Next, an operation of the health care system 30 is described.

### b4. Operation of Health Care System

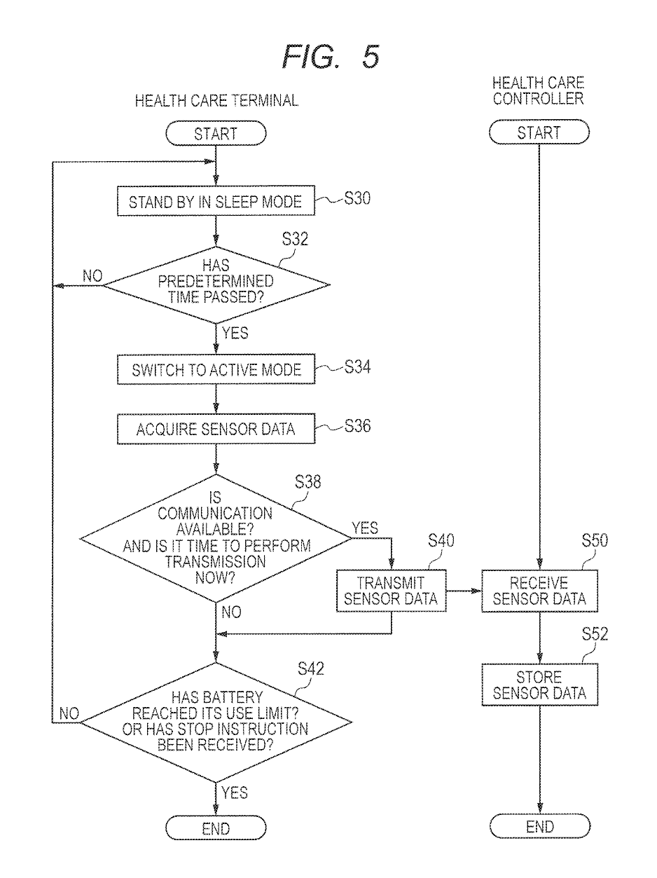

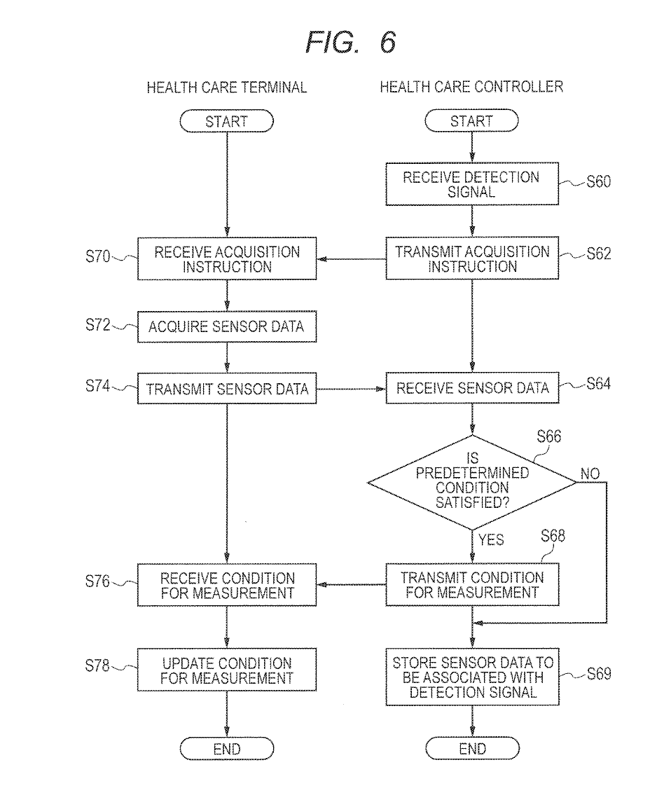

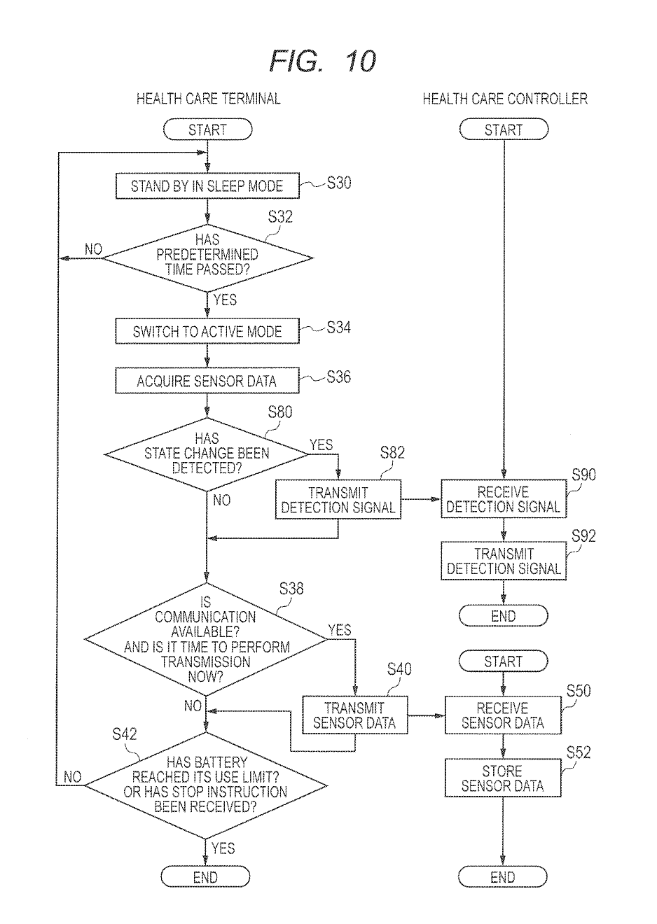

FIG. 5 is a flowchart of control of the health care system 30 according to the first embodiment. A process illustrated in FIG. 5 is achieved by execution of a control program by the health care terminal 300 and the health care controller 400. In another aspect, the process may be performed partially or entirely by a circuit element or other hardware. This can be also applied to FIGS. 6 and 10 described later.

Referring to FIG. 5, the health care terminal 300 stands by in a sleep mode in Step S30. The health care terminal 300 is configured to be switchable between an active mode in which acquisition of biological information by a biosensor is performed and the sleep mode in which an unnecessary function is stopped to reduce a power consumption as compared with that in the active mode. The health case terminal 300 suppresses the power consumption by cutting a power supply to the biosensor in the sleep mode. However, it is preferable that the activity meter 320 always performs measurement because of its characteristics that the activity meter 320 measures the amount of user's activity. Therefore, in another aspect, the health care terminal 300 may be configured to supply a power to the activity meter 320 also in the sleep mode.

In Step S32, the health care terminal 300 determines whether a predetermined time has passed after being switched to the sleep mode. The predetermined time is stored in the measurement condition storage unit 350, and is one minute, for example. When having determined that the predetermined time has passed (YES in Step S32), the health care terminal 300 proceeds to Step S34 and switches from the sleep mode to the active mode. On the other hand, when having determined that the predetermined time has not passed (NO in Step S32), the health care terminal 300 returns the process to Step S30.

In Step S36, the health care terminal 300 measures biological information by the biosensor, displays the measurement result on the display 360, and stores the measurement result in the data storing unit 365.

In Step S38, the health care terminal 300 determines whether communication with the health care controller 400 is available and it is a timing to perform transmission now. Specifically, the health care terminal 300 determines that communication with the health care controller 400 is available when communication has been established between the communication unit 310 and the communication unit 430. As an example of the transmission timing, the health care terminal 300 determines that it is the timing to perform transmission now, when the health care terminal 300 did not transmit the biological information to the health care controller 400 in the latest 24 hours.

When having determined that communication with the health care controller 400 is available and it is the timing to perform transmission now (YES in step S38), the health care terminal 300 transmits the biological information stored in the data storing unit 365 to the health care controller 400 (Step S40). The health care controller 400 receives the biological information from the health care terminal 300 in Step S50 and stores the received biological information in the data storage unit 420 in Step S52.

When having determined that communication with the health care controller 400 is not available or it is not the timing to perform transmission now (NO in Step S38), the health care terminal 300 proceeds to Step S42. In Step S42, the health care terminal 300 determines whether a battery has reached a use limit or a stop instruction has been received from the health care controller 400. For example, the health care terminal 300 determines that the battery has reached its use limit when the voltage of the battery falls to 3.3 V or lower.

When having determined that the battery has reached its use limit or the stop instruction has been received (YES in Step S42), the health care terminal 300 ends the process. On the other hand, when having determined that the battery has not reached its use limit and the stop instruction has not been received (NO in Step S42), the health care terminal 300 returns to Step S30, and switches to the sleep mode. The health care controller 400 manages user's health based on the biological information, such as the amount of activity, the body temperature, and the daily blood pressure acquired from the health care terminal 300. Next, control for updating a condition for measurement of the biosensor stored in the measurement condition storage unit 350 is described.

### b5. Update of Condition for Measurement

FIG. 6 is a flowchart of control for updating a condition for measurement of a biosensor according to the first embodiment.

In Step S60, the health care controller 400 receives the detection signal transmitted from the environment-monitor controller 200 in Step S22 (see FIG. 4). In Step S62, the health care controller 400 transmits an acquisition instruction instructing each biosensor to acquire biological information.

The health care terminal 300 receives the acquisition instruction from the health care controller 400 in Step S70, and acquires user's biological information (sensor data) by the biosensor in Step S72. In Step S74, the health care terminal 300 transmits the acquired biological information to the health care controller 400.

The health care controller 400 receives the biological information from the health care terminal 300 in Step S64, and determines whether the received biological information satisfies a predetermined condition in Step S66. The details of Step S66 will be described later.

When having determined that the received biological information satisfies the predetermined condition (YES in Step S66), the health care controller 400 transmits a condition for measurement of the biosensor to the health care terminal 300 (Step S68).

In Step S69, the health care controller 400 stores the received biological information (sensor data) in the data storage unit 420 to be associated with the detection signal. In other words, the health care controller 400 stores biological information measured in accordance with the condition for measurement stored in the measurement condition storage unit 350 and biological information measured in response to the detection signal in the data storage unit 420 separately from each other. In another aspect, the health care controller 400 may be configured to store environment information and the biological information in the data storage unit 420 to be associated with each other, in place of the detection signal. In that case, in Step S14, the environment-monitor controller 200 transmits the environment information together with the detection signal to the health care controller 400.

In Step S76, the health care terminal 300 receives the condition for measurement of the biosensor from the health care controller 400. In Step S78, the health care terminal 300 updates the condition for measurement stored in the measurement condition storage unit 350 by using the received condition for measurement.

According to the above, the sensor system 1 can detect a change of an external index (environment information) that can affect an object to be measured and can appropriately set a condition for measurement of the object to be measured based on a state of the object to be measured (a user) in accordance with the change of the external index. Therefore, the health care terminal 300 can efficiently acquire biological information useful to the user with a limited power, such as a battery.

### b6. Update of Condition for Measurement—Specific Example

A specific example of updating a condition for measurement of a biosensor is described below. A case where the condition for measurement of the biosensor is updated for taking measures against heat stroke is described as an example.

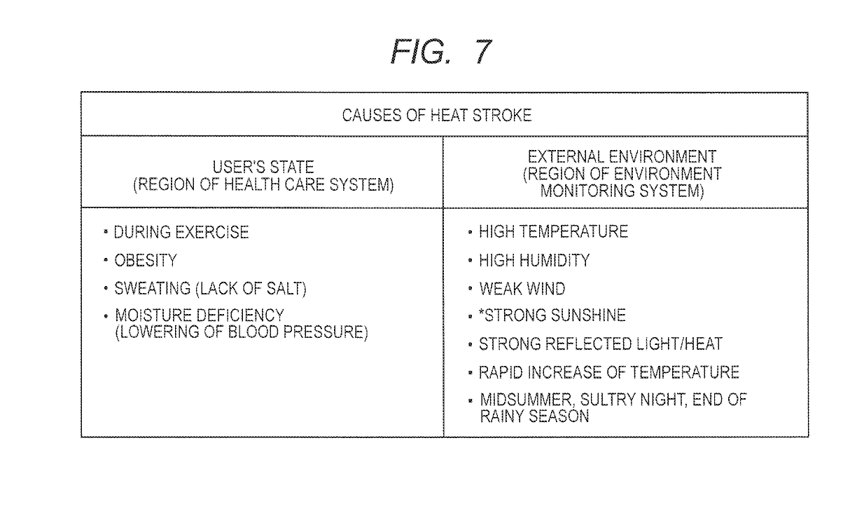

FIG. 7 illustrates causes of heat stroke. Referring to FIG. 7, factors causing heat stroke can be generally classified into ones caused by a state of a human being (a user of the health care terminal 300) and ones caused by an external environment.

As the causes of heat stroke, resulting from the external environment, a high temperature, a high humidity, and the like are considered. Therefore, in Step S12 in FIG. 4, the environment monitor terminal 100 determines that the change of the external environment has been detected, when either one of a condition that the temperature of the thermometer 150 exceeds 35° C. and a condition that the humidity of the hygrometer 160 exceeds 60% is satisfied.

On the other hand, moisture deficiency is considered as the cause of heat stroke resulting from the user's state. The moisture deficiency of the user wearing the health care terminal 300 can be observed as lowering of the blood pressure. Therefore, in Step S62 in FIG. 6, the health care controller 400 transmits an acquisition instruction instructing measurement for blood pressure of the user to the sphygmomanometer 330, in response to the detection signal from the environment-monitor controller 200. In response to the acquisition instruction, the sphygmomanometer 330 measures the blood pressure of the user separately from the condition for measurement stored in the measurement condition storage unit 350. The health care terminal 300 transmits the measurement result of the sphygmomanometer 330 measured in response to the detection signal, to the health care controller 400.

In Step S66, the health care controller 400 determines whether the blood pressure measured in response to the received detection signal is lower than a predetermined threshold value. For example, the health care controller 400 determines whether maximum blood pressure (systolic blood pressure) is lower than 100 mmHg. This threshold value is stored in the data storage unit 420. This threshold value is preferably a value based on medical knowledges. In further another aspect, this threshold value may be a value defined by gender and/or age of the user wearing the health care terminal 300.

When having determined that the blood pressure measured in response to the received detection signal is lower than the predetermined threshold value, the health care controller 400 transmits a condition for measurement that increases a measurement accuracy of the sphygmomanometer 330 in Step S68. This is because it is highly possible that the user wearing the health care terminal 300 gets heat stroke and it is necessary to monitor the user's state in more detail. As a control example of increasing the measurement accuracy, control for increasing a measurement frequency (a sampling rate) of a sensor, such as a sphygmomanometer, control for making a time spent for one measurement longer, and the like can be considered. As an example, the health care controller 400 selects one of a plurality of conditions for measurements stored in the data storage unit 420, that has a higher accuracy than a condition for measurement currently set, and transmits the selected condition for measurement to the health care terminal 300.

In another aspect, the health care controller 400 may be configured to decide the condition for measurement of the biosensor, further considering the measurement result of the activity meter 320 within a predetermined time period from reception of the detection signal. As illustrated in FIG. 7, a possibility of heat stroke increases during exercise. Therefore, when having determined that the user takes exercise at the time of reception of the detection signal based on the measurement result of the activity meter 320, the health care controller 400 can determine that a possibility that the user gets heat stroke is higher.

In Steps S76 and S78, the health care terminal 300 updates the condition for measurement stored in the measurement condition storage unit 350 by using the received condition for measurement. In another aspect, the health care terminal 300 may be configured to display an image of warning that there is a risk of heat stroke on the display 360 in response to reception of the condition for measurement from the health care controller 400.

According to the above, the sensor system 1 can improve a measurement accuracy of a biosensor in order to monitor a user's state in more detail, when a user of the health care terminal 300 is placed in a dangerous state in accordance with a change of environment information, regardless of whether the user is aware or unaware of that state. That is, the sensor system 1 can optimize the health care terminal 300 for each user.

### b7. Update of Condition for Measurement—First Modified Example

The sensor system 1 is configured to update a condition for measurement stored in the measurement condition storage unit 350 based on a measurement result measured in response to a detection signal in the above example, but is not limited thereto. In another aspect, the sensor system 1 may be configured to update the condition for measurement based on a measurement result measured in accordance with the condition for measurement stored in the measurement condition storage unit 350 (hereinafter, also referred to as “normal data”) and the measurement result measured in response to the detection signal (hereinafter, also referred to as “sudden data”).

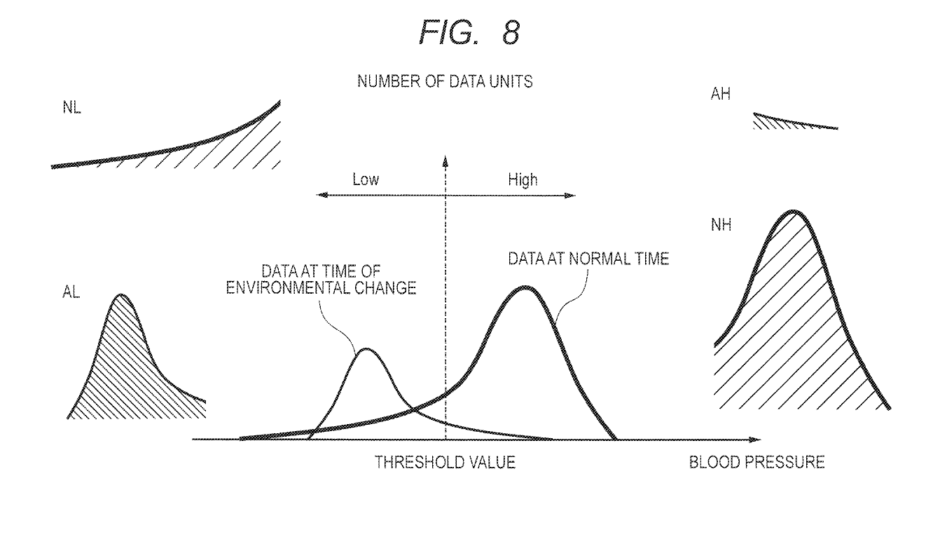

FIG. 8 illustrates another control example in Step S66 in FIG. 6. In FIG. 8, normal data stored in the data storage unit 420 and sudden data are represented by distributions of the number of data units for each blood pressure value (for example, a maximum blood pressure value).

As illustrated in FIG. 8, the number of data units of blood pressure stored in the data storage unit 420 is a value obtained by adding the number of normal data units less than a predetermined threshold value (hereinafter, also referred to as “NL”), the number of normal data units equal to or more than the threshold value (hereinafter, also referred to as “NH”), the number of sudden data units less than the threshold value (hereinafter, also referred to as “AL”), and the number of sudden data units equal to or more than the threshold value (hereinafter, also referred to as “AH”).

The health care controller 400 calculates an index A indicating a possibility that the user of the health care terminal 300 gets heat stroke by the following Expression (1).

$$A = \frac{NH + AL}{NH + NL + AH + AL} \qquad ( 1 )$$

In the health care controller 400, the distribution of the normal data units with respect to the threshold value and the distribution of the sudden data units with respect to the threshold value are more different from each other, as a value of the index A is larger. The health care controller 400 determines that a possibility that the user gets heat stroke is higher as the value of the index A is larger. As an example, the health care controller 400 determines that the predetermined condition in Step S66 in FIG. 6 is satisfied, when the index A is 0.8 or more.

According to the above, the sensor system 1 can change a condition for measurement based on statistical data including normal data and sudden data. The sensor system 1 with this configuration can determine a possibility of getting heat stroke with a higher accuracy, as compared with a configuration that determination is made only based on a measurement result measured in response to a received detection signal.

Note that the number of normal data units and the number of sudden data units are largely different from each other in many cases. Therefore, the health care controller 400 may set the time spent for one measurement of the sphygmomanometer 330 to be longer so that both the numbers can be compared as statistical data in step S68 in FIG. 6.

### b8. Update of Condition for Measurement—Second Modified Example

The sensor system 1 has a configuration that takes measures against a known symptom (heat stroke), that is, a configuration that grasps in advance a biosensor of which a condition for measurement is changed in the above example. In another aspect, the sensor system 1 may be configured to take measures against an unknown symptom. This configuration is described below.

When having received a detection signal from the environment-monitor controller 200, the health care controller 400 performs measurement for biological information by all biosensors mounted in the health care terminal 300 until normal data and sudden data can be compared as statistical data. As an example, until the number of data units of sudden data related to certain biological information reaches a predetermined number (e.g., 100), the health care controller 400 acquires biological information by all the biosensors.

When the number of data units of the sudden data has reached the predetermined number, the health care controller 400 calculates the index A represented by the above Expression (1) with regard to each biological information. The health care controller 400 changes a condition for measurement of a biosensor corresponding to biological information for which the index A is equal to or larger than a predetermined value (e.g., 0.8). After the number of data units of the sudden data has reached the predetermined number, the health care controller 400 performs measurement only by the biosensor of which the condition for measurement has been changed, in response to reception of the detection signal.

According to the above, also as for an unknown symptom, the sensor system 1 can also change a condition for measurement of a biosensor based on a change of a user's state in accordance with a change of an external environment. Further, the sensor system 1 does not acquire biological information that is not related to the change of the external environment after the condition for measurement is changed, even when the detection signal is received.

### b9. Plural Health Care Terminals 300

In the above example, the health care terminal 300 that communicates with the health care controller 400 is one. In another aspect, the health care controller 400 may be configured to communicate with a plurality of health care terminal 300. For example, the health care controller 400 is provided in a house and family members living in that house respectively wear the health care terminals 300. In another example, the health care controller 400 is provided in an office, and employees respectively wear the health care terminals 300.

In that case, the predetermined threshold value used in determination in Step S66 in FIG. 6 may be an average value of biological information obtained from all the health care terminals 300 coupled to the health care controller 400.

### C. Second Embodiment

As the sensor system 1 according to the first embodiment, the configuration is described by way of a measure for heat stroke as an example, in which a detection signal is transmitted from the environment monitoring system 10 to the health care system 30. A sensor system according to a second embodiment also transmits the detection signal from a health care system to an environment monitoring system. That is, while using a state (biological information) of a user wearing a health care terminal as an external index, the sensor system 1A also measures environment information in accordance with a change of this state. The configuration and control of the sensor system 1A are described below. Note that because the basic configuration of the sensor system 1A is approximately the same as the sensor system 1, only differences are described.

### c1. Configuration of Sensor System 1A

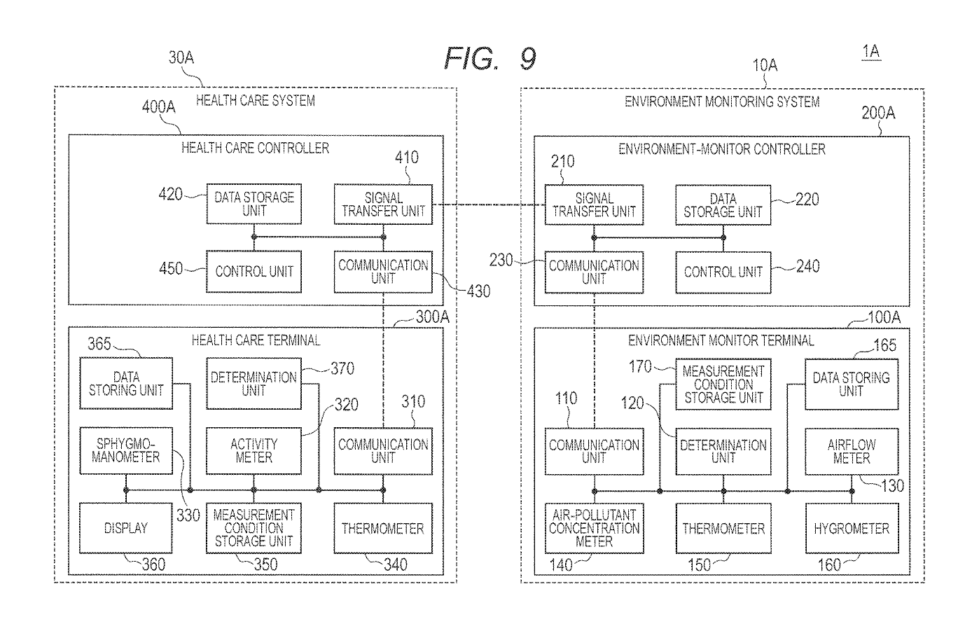

FIG. 9 illustrates a configuration example of the sensor system 1A according to the second embodiment. Referring to FIG. 9, the sensor system 1A includes an environment monitoring system 10A and a health care system 30A. The environment monitoring system 10A includes an environment monitor terminal 100A and an environment-monitor controller 200A. The health care system 30A includes a health care terminal 300A and a health care controller 400A.

The environment monitor terminal 100A further includes a measurement condition storage unit 170, as compared with the environment monitor terminal 100. The measurement condition storage unit 170 stores therein various conditions for measurement of an environment sensor.

The health care terminal 300A further includes a determination unit 370, as compared with the health care terminal 300. The determination unit 370 determines whether biological information received from a biosensor satisfies a predetermined condition, and outputs a detection signal based on a change of the biological information (hereinafter, also referred to as a “biological detection signal”) to the health care controller 400A via the communication unit 310, when having determined that the biological information satisfies that condition.

### c2. Operation of Health Care System 30A

FIG. 10 is a flowchart of control of the health care system 30A according to the second embodiment. Because portions labeled with the same reference signs as those in FIG. 5 are the same portions, the description thereof is omitted.

In Step S80, the health care terminal 300A determines whether a change of user's state has been detected based on acquired biological information (sensor data). More specifically, the health care terminal 300A determines whether the acquired biological information satisfies a predetermined condition.

When having determined that the acquired biological information satisfies the predetermined condition (YES in Step S80), the health care terminal 300A transmits a biological detection signal to the health care controller 400A in Step S82. On the other hand, when having determined the acquired biological information does not satisfy the predetermined condition (NO in Step S80), the health care terminal 300A proceeds to Step S38.

In Step S90, the health care controller 400A receives the biological detection signal from the health care terminal 300A. In Step S92, the health care controller 400A transmits the received biological detection signal to the environment monitoring system 10A. The processes in Steps S90 and S92 in the health care controller 400A are so-called interrupt processing, and are different from a normal process. The health care controller 400A transmits the detection signal in Step S92, and then ends a series of interrupt processing. Thereafter, the health care controller 400A returns to the normal process and waits for reception of the biological information from the health care terminal 300A.

### c3. Update of Condition for Measurement of Environment Sensor

FIG. 11 is a flowchart of control for updating a condition for measurement of an environment sensor according to the second embodiment.

In Step S100, the environment-monitor controller 200A receives the biological detection signal transmitted from the health care controller 400A in Step S92 (see FIG. 7). In Step S102, the environment-monitor controller 200A transmits an acquisition instruction instructing each environment sensor to acquire environment information in response to reception of the biological detection signal.

The environment monitor terminal 100A receives the acquisition instruction from the environment-monitor controller 200A in Step S120, and acquires environment information (sensor data) by the environment sensor in Step S122. In Step S124, the environment monitor terminal 100A transmits the acquired environment information to the environment-monitor controller 200A.

The environment-monitor controller 200A receives the environment information from the environment monitor terminal 100A in Step S104, and determines whether the received environment information satisfies a predetermined condition in Step S106.

When having determined that the received environment information satisfies the predetermined condition (YES in Step S106), the environment-monitor controller 200A transmits a condition for measurement of the environment sensor to the environment monitor terminal 100 (Step S108).

In Step S109, the environment-monitor controller 200A stores the received environment information (sensor data) in the data storage unit 220 to be associated with the biological detection signal. In other words, the environment-monitor controller 200A stores environment information measured in accordance with the condition for measurement stored in the measurement condition storage unit 170 and environment information measured in response to the biological detection signal in the data storage unit 220 separately from each other. In another aspect, the environment-monitor controller 200A may be configured to store biological information and the environment information in the data storage unit 220 to be associated with each other, in place of the biological detection signal. In that case, in Step S82, the health care controller 400A transmits the biological information together with the biological detection signal to the environment-monitor controller 200A.

In Step S126, the environment monitor terminal 100A receives the condition for measurement of the environment sensor from the environment-monitor controller 200A. In Step S128, the environment monitor terminal 100A updates the condition for measurement stored in the measurement condition storage unit 170 by using the received condition for measurement.

According to the above, the sensor system 1A according to the second embodiment can detect a change of biological information as an external index, and can appropriately set a condition for measurement of an environment sensor based on environment information in accordance with the change of the external index.

A specific example of updating the condition for measurement of the environment sensor is described below. As an example, a case where a condition for measurement of the environment sensor is updated for preventing hey fever is described. In this specific example, the health case terminal 300A is a terminal in form of an adhesive plaster, and is worn to the chest of a user. Also, in this specific example, the active meter of the health care terminal 300A is assumed to continue measurement also in the sleep mode.

The health care terminal 300A detects a sneeze and a cough from a signal waveform of an acceleration sensor mounted in the active meter 320. More specifically, the active meter 320 stores signal patterns of the acceleration sensor that correspond to the sneeze and the cough in a storage device (not illustrated). The active meter 320 detects the sneeze and the cough, when having detected a portion corresponding to those signal patterns from a measurement result of the acceleration sensor.

The health care terminal 300A determines that a change of a user's state has been detected, when having detected the sneeze and the cough based on the measurement result of the active meter 320 in Step S80 in FIG. 10, and transmits a biological detection signal to the health care controller 400A. In another aspect, the health care terminal 300A may be configured to transmit the biological detection signal when the detected number of the sneeze or the cough within a unit time exceeds a predetermined number based on the measurement result of the active meter 320.

In Steps S100 and S102 in FIG. 11, the environment-monitor controller 200A transmits an acquisition instruction that instructs measurement of an air-pollutant concentration to the air-pollutant concentration meter 140 in response to reception of the biological detection signal from the health care controller 400A.

In response to this, in Steps S122 and S124, the air-pollutant concentration meter 140 measures an air-pollutant concentration in surroundings separately from a condition for measurement stored in the measurement condition storage unit 170, and transmits the measurement result to the environment-monitor controller 200A.

In Step S106, the environment-monitor controller 200A determines whether the counts of pollen per unit volume is a predetermined threshold value or more based on the received measurement result. For example, the air-pollutant concentration meter counts the number of minute particles per unit volume that have been classified by diameter. The environment-monitor controller 200A determines minute particles with a diameter of 20 to 40 μm as pollen. For example, the environment-monitor controller 200A determines whether the counts of pollen per cubic meter is 12 or more. This predetermined threshold value is stored in the data storage unit 220. This threshold value is preferably based on medical knowledge.

When having determined that the counts of pollen per unit volume is a predetermined number or more, the environment-monitor controller 200A transmits a condition for measurement that increases a measurement accuracy of the air-pollutant concentration meter 140 to the environment monitor terminal 100A in Step S108.

In another aspect, in Step S108, the environment-monitor controller 200 may be configured to further transmit an instruction that makes the display 360 of the health care terminal 300A display an image of warning that a surrounding pollen concentration is high, to the health care controller 400A.

In further another aspect, in Step S108, the environment-monitor controller 200A may be configured to transmit a control signal for removing pollen, to an external device, such as an air cleaner (not illustrated). With this configuration, the sensor system 1A can remove pollen actively from a stage in which there is no subjective symptom to hey fever in a user or a state in which the symptoms are not serious, thereby preventing hey fever from becoming serious.

In Steps S126 and S128, the environment-monitor terminal 100A updates a condition for measurement stored in the measurement condition storage unit 170 by using the received condition for measurement.

According to the above, the sensor system 1A can appropriately update a condition for measurement of an environment sensor automatically in accordance with a state change of a user of the health care terminal 300A. That is, the sensor system 1A can optimize the condition for measurement of the environment sensor for every user.

### D. Third Embodiment

The sensor systems according to the above embodiments have a configuration in which the systems each including the sensor terminal and the controller communicate with each other. A sensor system 1B according to a third embodiment employs a configuration in which the sensor terminals communicate with each other. When the basic configuration of the sensor system 1B is described, only differences from the sensor system 1 are described.

### d1. Configuration of Sensor System 1B

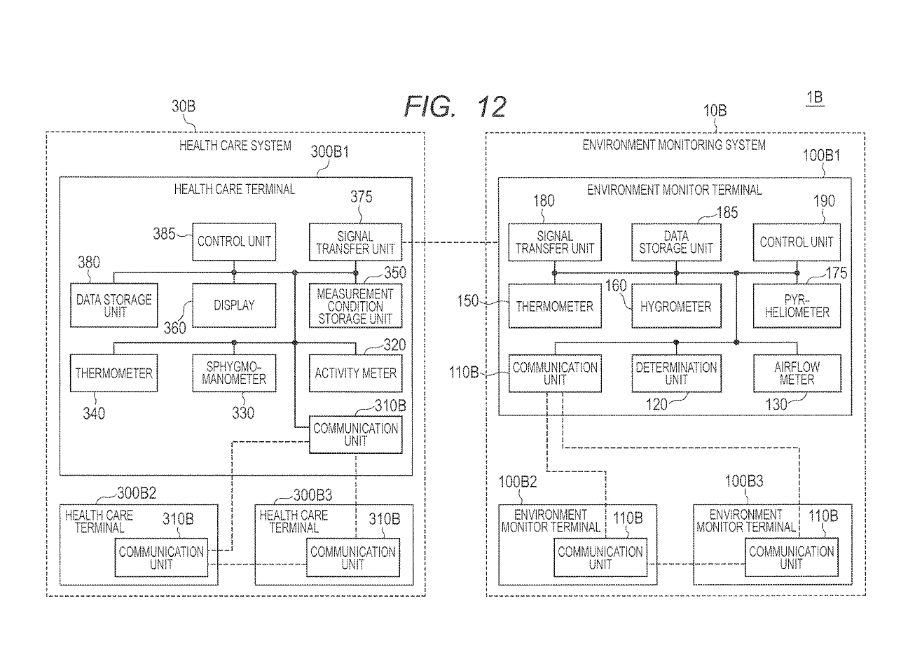

FIG. 12 illustrates a configuration example of the sensor system 1B according to the third embodiment. The sensor system 1B includes an environment monitoring system 10B and a health care system 30B. The environment monitoring system 10B includes a plurality of environment monitor terminal 100B1, 100B2, 100B3, . . . (hereinafter, also collectively referred to as an “environment monitor terminal 100B”). The health care system 30B includes a plurality of health care terminals 300B1, 300B2, 300B3, . . . (hereinafter, also collectively referred to as a “health care terminal 300B”).

The environment monitor terminal 100B includes a pyrheliometer 175 in place of the air-pollutant concentration meter 140 therein and has various functions mounted on the environment-monitor controller 200. Specifically, the environment monitor terminal 100B further includes a signal transfer unit 180, a data storage unit 185, and a control unit 190. The signal transfer unit 180 is configured to be communicable with the health care terminal 300B. The data storage unit 185 stores therein a measurement result (environmental information) of an environment sensor (the airflow meter 130, the thermometer 150, the hygrometer 160, and the pyrheliometer 175. The control unit 190 performs predetermined processing based on the environment information stored in the data storage unit 185 to determine a condition for measurement of the environment sensor. Further, the communication unit 110B is configured to be communicable with another environment monitor terminal 100B.

The health care terminal 300B further includes various functions mounted on the health care controller 400. Specifically, the health care terminal 300B includes a signal transfer unit 375, a data storage unit 380, and a control unit 385. The data storage unit 380 stores therein a measurement result (biological information) of a biosensor. Further, the communication unit 310B is configured to be communicable with another health care terminal 300B.

The health care terminal 300B has a function of communicating with the environment monitor terminal 100B, in addition to a function of acquiring the biological information. Therefore, the health care terminal 300B does not have to perform communication with the health care controller, which has been described in the above embodiments. That is, a user wearing the health care terminal 300B can freely move without being restricted in a region where communication with the health care controller is available. Further, the health care terminal 300B also has a data processing function, it can perform various types of real-time processing using the biological information.

### d2. Control in Sensor System 1B

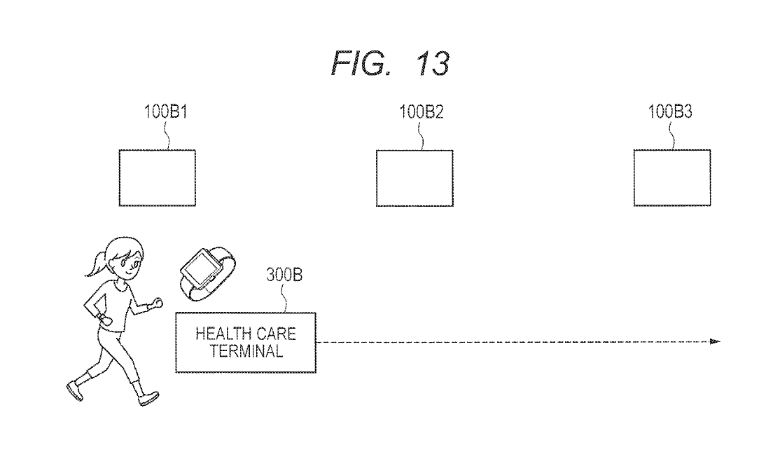

FIG. 13 illustrates a use example of the sensor system 1B according to the third embodiment. As illustrated in FIG. 13, a user wearing the health care terminal 300B jogs on a road on which the environment sensor terminals 100B are arranged not to be movable. In this use example, the sensor system 1B prevents heat stroke.

Each of the environment monitor terminals 100B calculates Wet Bulb Globe Temperature WBGT from a measurement result of an environment sensor. When the Wet Bulb Globe Temperature WBGT exceeds a predetermined value (e.g., 25° C.), the environment monitor terminal 100B determines that an environmental change has been detected, and searches a communicable health care terminal 300B therearound, The environment monitor terminal 100B transmits a detection signal to the health care terminal 300B with which communication has been established, which indicates detection of the environmental change.

In the example illustrated in FIG. 13, when the user jogs, the environment monitor terminal 100B that transmits the detection signal to the health care terminal 300B is switched from the environment monitor terminal 100B1 to the environmental monitor terminal 100B2, and then to the environmental terminal 100B3 in that order as time passes.

With this configuration, even when the user moves, the health care terminal 300B can receive a highly accurate risk notification (the detection signal) of heat stroke (an abnormal state of the user) based on accurate environment information at the destination of the user′ move from the environment monitor terminal 100B installed at the destination.

In addition, the health care terminal 300B changes a condition for measurement for the biological information, such as blood pressure or body temperature, in real time in accordance with reception of the detection signal from the environment monitor terminal 100B, and monitors the user's state in detail. When the user's state worsens, the health care terminal 300B notifies the display 360 of that fact. Therefore, the sensor system 1B enables the user to avoid heat stroke.

### d3. Link with Environment Monitor Terminal 100B

The environment monitor terminal 100B may be configured to share environment information with another environment monitor terminal 100B via the communication unit 110B. With this configuration, the environment monitor terminal 100B can present a place where there is a high possibility of causing a state abnormality (for example, heat stroke) to a user based on environment information acquired by the other environment monitor terminal 100B as an example. A configuration may be employed in which the environment monitor terminal 100B transmits environment information to a server (not illustrated) and the server manages the environment information.

In another example, the environment monitor terminal 100B may be configured to transmit a signal indicating that it has transmitted a detection signal to the health care terminal 300B, to another environment monitor terminal 100B. For example, the environment monitor terminal 100B that has received that signal may limit transmission of the detection signal to the health care terminal 300B for a predetermined period.

### d4. Link with Health Care Terminal 300B

The health care terminal 300B may be configured to establish communication with the environment monitor terminal 100B via another health care terminal 300B. With this configuration, even in a case where there is no environment monitor terminal 100B around the health care terminal 300B, with which the health care terminal 300B can directly communicate, the health care terminal 300B can receive a risk notification (a detection signal) related to a state abnormality of a user via another health care terminal 300B.

Further, the health care terminal 300B can calculate an average value of biological information of users each wearing the health care terminals 300B by sharing the biological information with another health care terminal 300B. For example, the health care terminal 300B may be configured to update a condition for measurement when a difference between the biological information of the user and the average value exceeds a predetermined value. Furthermore, a configuration may be employed in which the health care terminal 300B transmits the biological information to a server (not illustrated) and the server manages the biological information.

The sensor systems according to the first to third embodiments described above are configured to acquire environment information (or biological information) as an external index, acquire biological information (or environment information) as an object to be measured based on a change of the external index, and change a condition for measurement of the object to be measured based on a measurement result, but are not limited thereto. As another example, the sensor system can be also used for improving productivity/safety in a factory. Specifically, a worker in the factory wears a wearable terminal (e.g., smart glasses) with various sensors mounted thereon. Also, a system that grasps an operation status of the factory is configured. In such an environment, a configuration may be employed in which measurement by the sensor mounted on the wearable terminal is performed in accordance with a change of the operation status as an external index, and a condition for measurement of the sensor is updated based on the measurement result.

In the above description, the invention made by the inventors of the present application has been specifically described by way of the embodiment. However, the present invention is not limited to the aforementioned embodiments, and can be changed in various ways within the scope not departing from the gist thereof.

What is claimed is:

1. A sensor system comprising:
first and second sensors;
a determination unit for outputting a detection signal when a measurement result of the first sensor satisfies a predetermined condition;
a measurement condition storage unit for storing a condition for measurement of the second sensor, and
a control unit for performing measurement by the second sensor separately from measurement in accordance with the condition for measurement, when having received the detection signal, and for updating the condition for measurement of the second sensor stored in the measurement condition storage unit based on a result of the performed measurement.

2. The sensor system according to claim 1,
wherein the condition for measurement of the second sensor stored in the measurement condition storage unit includes at least one of a measurement frequency and a time spent for measurement.

3. The sensor system according to claim 1,
wherein one of the first and second sensors is configured to be unmovable, and the other one is configured to be movable.

4. The sensor system according to claim 3,
wherein the other sensor is configured to be capable of being worn by a human body.

5. The sensor system according to claim 3,
wherein the one sensor acquires environment information, and the other sensor acquires biological information.

6. The sensor system according to claim 1, further comprising a storage device for storing a first measurement result of the second sensor, measured in accordance with the condition for measurement stored in the measurement condition storage unit, and a second measurement result of the second sensor, measured in response to reception of the detection signal, separately from each other,
wherein the control unit updates the condition for measurement of the second sensor stored in the measurement condition storage unit based on the first measurement result and the second measurement result stored in the storage device.

7. The sensor system according to claim 6, wherein the control unit updates the condition for measurement of the second sensor stored in the measurement condition storage unit based on a number of the first measurement results below a predetermined threshold value, a number of the second measurement results below the predetermined threshold value, a number of the first measurement results equal to or more than the predetermined threshold value, and a number of the second measurement results equal to or more than the predetermined threshold value.

8. The sensor system according to claim 1,
wherein the control unit transmits a control signal to an external device based on the measurement result of the second sensor measured in response to reception of the detection signal.

9. The sensor system according to claim 1,
wherein a plurality of first sensor terminals each including the first sensor are provided, and
wherein each of the first sensor terminals includes a communication unit for communicating with another one of the first sensor terminals.

10. The sensor system according to claim 1,
wherein a plurality of second sensor terminals each including the second sensor are provided, and
wherein each of the second sensor terminals includes a communication unit for communicating with another one of the second sensor terminals.

11. A sensor system comprising:
a sensor;
a measurement condition storage unit for storing a condition for measurement of the sensor; and
a control unit for performing measurement by the sensor separately from measurement in accordance with the condition for measurement, when having received a predetermined signal from an external device, and for updating the condition for measurement of the sensor stored in the measurement condition storage unit based on a result of the performed measurement.

12. A sensor system comprising:
a first sensor;
a determination unit that determines whether a measurement result of the first sensor satisfies a predetermined condition, and
a communication unit for transmitting to another system including a second sensor a detection signal for changing a condition for measurement of the second sensor, when the predetermined condition is determined to be satisfied.
